(19) United States
(12) Patent Application Publication
(10) Pub. No.: US 2003/0124726 A1
(43) Pub. Date: Jul. 3, 2003

(54) Global expression analysis of extracellular matrix-integrin interactions in monocytes

(76) Inventors: Antonin deFougerolles, Brookline, MA (US); Philip Gotwals, Needham, MA (US); Cynthia Green, Madison, CT (US); Victor Koteliansky, Boston, MA (US)

Correspondence Address: MINTZ, LEVIN, COHN, FERRIS,, GLOVSKY AND POPEO, P.C., One Financial Center, Boston, MA, 02111, US

(21) Appl. No.: 09/924,168

(22) Filed: Aug. 3, 2001

Related U.S. Application Data
(60) Provisional application No. 60/222,874, filed on Aug. 3, 2000.

(51) Int. Cl. C12N 15/87; A61K 48/00

(52) U.S. Cl. 435/455; 514/44

(57) ABSTRACT

Disclosed are methods of identifying integrin modulating agents using differential gene expression. Also disclosed are method of treating atheroscerosis and inflammatory disorders in a subject.

### RELATED U.S. APPLICATION

This application claims priority to U.S. Ser. No. 60/222,874, filed Aug. 3, 2000 which is incorporated herein by reference in its entirety.

### FIELD OF THE INVENTION

The invention relates generally to nucleic acids and polypeptides and in particular to the identification of integrin modulating agents using differential gene expression.

### BACKGROUND OF THE INVENTION

Integrins are cell surface receptors that are evolutionarily conserved molecules found in a wide variety of cell types. They are composed of two subunits, an alpha and a beta subunit, and there are many varieties of each subunit. These subunits are are mixed and matched in various cell types to form specialized receptors that have unique binding specificity and signaling capabilities. Specialized signaling via integrins effects the activities of cytoplasmic kinases, growth factor receptors, ion channels and organization of the intracellular cytoskeletal components. These activities can direct cell proliferation, differentiation, or apoptosis, cell fates which are important in many aspects of normal growth as well as disease. Giancotti and Ruoslahti, 1999 Science 285:1028-1032. Integrins are also important for optimal function of other membrane receptors such as PDGF, EGF and VEGF, under certain attachment conditions.

### SUMMARY OF THE INVENTION

The invention is based in part on the discovery that certain nucleic acids are differentially expressed in monocytes when exposed to various extracellular matrix components in the presence or absence of growth factors.

In further aspect, the invention provides a method of screening a test agent for integrin modulating activity. For example, in one aspect, the invention provides a method of identifying a integrin modulating agent by providing a test cell population comprising a cell capable of expressing one or more nucleic acids sequences responsive to integrin modulators, contacting the test cell population with the test agent and comparing the expression of the nucleic acids sequences in the test cell population to the expression of the nucleic acids sequences in a reference cell population not treated with an integrin modulators. An alteration in expression of the nucleic acids sequences in the test cell population compared to the expression of the gene in the reference cell population indicates that the test agent is an integrin modulator.

Also included in the invention is a method of treating or preventing atherosclerosis in a subject the method by administering to the subject an agent that modulates the expression or activity of one or more of the FNX nucleic acids.

The invention also provides a method treating or preventing an inflammatory disorder in a subject, by administering to the subject an agent that modulates the expression or activity of one or more of the FNX nucleic acids selected from the group consisting of FNX1-208 and 209.

In a further aspect, the invention includes a method of inhibiting monocyte differention in by contacting the cell with an agent that modulates the expression or activity of one or more of the FNX nucleic acids.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1. Distribution by fold change of differentially expressed fragments upon attachment to extracellular matrix molecules. Cells attached fibronectin (FN), collagen type I (CO I), or laminin (LM) were compared to cells left in suspension, under conditions listed in Table 1. The number of differentially expressed fragments are grouped by fold change (solid bars: upregulated fragments; grey bars: downregulated fragments). FIG. 2. The effect of growth factors on fibronectin-mediated attachment activation of NF-kB and STAT1. Fibronectin-mediated activation of NF-kB occurs irrespective of the presence of growth factors, while activation of STAT1 requires the presence of growth factors. (A). Induction of NF-kB-mediated transcription. THP-1 cells were transfected with a kB luciferase reporter construct (2×kB sites) and adhered to plates containing immobilised fibronectin for 2 and 4 hrs in either the presence of serum-free media or media containing 2% fetal bovine serum. Cells were collected and luciferase activity was measured. Results are normalized to luciferase activity seen in transfected cells left in suspension. (B). Induction of NF-kB translocation into the nucleus. Coverslips were plated with either fibronectin or poly-lysine control substrate. THP-1 cells were attached for 4 hrs in serum-free media or media containing 2% fetal bovine serum, washed, and fixed in paraformaldehyde. Fixed cells were then permeabilized using 0.2% Triton X-100 and stained for NF-kB p65 using a rabbit anti-NF-kB p65 antisera followed by a FITC-donkey anti-rabbit secondary reagent. Slides were mounted and NF-kB staining was visualized under fluorescent microscopy. (C). Effect of growth factors on fibronectin-mediated activation of STAT1. THP-1 cells were starved for 72 hours, and adhered to plates containing immobilised fibronectin for the indicated times in either the presence of serum-free media or media containing 2% fetal bovine serum. Bound cells were lysed and cell extracts were subjected to 8% SDS-PAGE. Proteins were electrophoretically transferred to nitrocellulose filter and immunoblotted with either a rabbit anti-phosphoSTAT1 (Tyr 701) antibody to detect activated STAT1 or a rabbit anti-STAT1 polyclonal antisera to detect total STAT1. (D)

FIG. 3. Comparison of gene regulation through attachment to different integrin ligands. Venn diagram illustration of the number of differentially expressed fragments (compared to cells in suspension) induced upon 1 hr attachment to either collagen type I, laminin, or 120 kD fragment of fibronectin.

### DETAILED DESCRIPTION OF THE INVENTION

The present invention is based in part on the discovery of changes in expression patterns of multiple nucleic acid sequences in the human monocytic cell line, THP-1 following exposure to various integrin modulating agents. The intergin modulating agents included extracellular matrix components, e.g., fibronectin, type I collagen, laminin and VCAM.

The differentially expressed nucleic acids were identified by culturing the THP-1 cells either adherently or in suspension in the presence of the integrin modulating agent. Control cultures received poly-lysine. Following treatment cells were lysed and total RNA was recovered from the lysed cells. cDNA was prepared and the resulting samples were processed through using GENECALLING™ differential expression analysis as described in U. S. Pat. No. 5,871,697 and in Shimkets et al., Nature Biotechnology 17:798-803 (1999). The contents of these patents and publications are incorporated herein by reference in their entirety.

1189 of gene fragments were initially found to be differentially expressed in THP-1 cells in response to integrin modulating agents. Genes fragments whose expression levels were modulated greater than ±5.0-fold were selected for further analysis.

A summary of the sequences analyzed are presented in Tables 2, 3 and 5. The 209 single nucleic acid sequences identified herein, are referred to herein as FNX 1-209.

For a given FNX sequence, its expression can be measured using any of the associated nucleic acid sequences may be used in the methods described herein. For previously described sequences database accession numbers are provided. This information allows for one of ordinary skill in the art to deduce information necessary for detecting and measuring expression of the FNX nucleic acid sequences.

TABLE 2
THP-1 cell attachment to fibronectin activates the NF-κB and
interferon pathways of gene induction. The names of the
differentially expressed genes are given with their GenBank
accession number, the number of fragments that were identified,
and the average fold induction of the gene. Genes linked to the
NF-κB pathway are shown in green and those linked to the
Jak/STAT pathway are shown in red.

| | GenBank | | Fold | FNX |
|---|---|---|---|---|
| Encoded Protein | Acc. No. | Fragments | Induction | No |
| Secreted Factors | | | | |
| MCP-1 | S69738 | 2 | 38.5 | 1 |
| FB13 | L08187 | 2 | 30.1 | 2 |
| TNFα | X01394 | 1 | 28.5 | 3 |
| IL-B30 | AA418955 | 1 | 27.7 | 4 |
| IL-8 | M28130 | 2 | 14.5 | 5 |
| GRO-α | J03561 | 1 | 13.8 | 6 |
| ISG15 | AH001495 | 3 | 12.0 | 7 |
| PBEF | U02020 | 2 | 11.1 | 8 |
| IP10 | X02530 | 3 | 10.1 | 9 |
| MIP-1α | D00044 | 2 | 9.6 | 10 |
| IL-1β | M15330 | 3 | 7.8 | 11 |
| RANTES | M21121 | 1 | 4.7 | 12 |
| Cartilage gp 39 | Y08378 | 1 | 4.4 | 13 |
| GRO-β | M36820 | 1 | 3.1 | 14 |
| IL-1ra | AF043143 | 1 | 2.4 | 15 |
| Cell Surface Proteins | | | | |
| CD44 | M69215 | 1 | 18.7 | 16 |
| IL10 receptor | U00672 | 1 | 13.2 | 17 |
| CD40 | X60592 | 1 | 9.7 | 18 |
| Leu-13 (9-27) | J04164 | 1 | 3.4 | 19 |
| CD82 | X53795 | 1 | 2.8 | 20 |
| CD18 | M15395 | 1 | 2.6 | 21 |
| Tissue Factor | M16553 | 1 | 2.2 | 22 |
| Metabolic Proteins | | | | |
| MnSOD | X65965 | 2 | 21.0 | 23 |
| Ribosomal protein S20 | L06498 | 1 | 10.5 | 24 |
| Long-chain acyl-CoA | D10040 | 2 | 9.0 | 25 |
| synthetase 1 | | | | |
| Spermidine/spermine | M77693 | 1 | 8.7 | 26 |
| N1-acetyltransferase | | | | |
| Ferritin Heavy chain | L20941 | 1 | 8.4 | 27 |
| γ-Glutamylcysteine synthetase | M90656 | 2 | 6.5 | 28 |
| catalytic subunit (GLCLC) | | | | |
| FNXphilin | X97324 | 1 | 4.5 | 29 |
| Phosphogluconate | U30255 | 1 | 4.1 | 30 |
| dehydrogenase | | | | |
| Kynureninase | U57721 | 1 | 3.0 | 31 |
| Transcription Factors | | | | |
| STAT5A | L41142 | 2 | 17.0 | 32 |
| BC1.3 | M31732 | 1 | 11.3 | 33 |
| NFκB1 p105/p50 | Z47744 | 2 | 7.9 | 34 |
| LITAF (PIG7) | U77396 | 1 | 7.7 | 35 |
| RelB (I-Rel) | M83221 | 1 | 3.8 | 36 |
| JUN-B (G0S3) | M29039 | 1 | 2.8 | 37 |
| PU.1 | NM003120 | 1 | 2.5 | 38 |
| ATF3 | H03023 | 1 | 2.4 | 39 |
| IRF1 | L05072 | 1 | 2.1 | 40 |
| Regulatory Proteins | | | | |
| IF156 | X03557 | 1 | 38.4 | 41 |
| TNFAIP2 (B94) | M92357 | 2 | 20.0 | 42 |
| c-src | X03996 | 1 | 13.6 | 43 |
| p62 lck ligand | U46751 | 1 | 9.9 | 44 |
| G0S2 | M69199 | 1 | 7.7 | 45 |
| Annexin VIII | X16662 | 1 | 7.4 | 46 |
| p59 Oligoadenylate | AJ225089 | 2 | 5.9 | 47 |
| Synthetase-like prot. | | | | |
| Na/K-transporting | M25161 | 1 | 5.7 | 48 |
| ATPase β1 chain | | | | |
| Fascin | U09873 | 1 | 5.7 | 49 |
| Annexin II | M14043 | 1 | 5.4 | 50 |
| sim Cyclophilin C | R32012 | 2 | 5.2 | 51 |
| sim PIM-1 | AA504311 | 1 | 5.2 | 52 |
| IF141 | L22342 | 1 | 4.7 | 53 |
| MyD88 | U70451 | 1 | 4.6 | 54 |
| CsA-binding protein | AI471997 | 1 | 4.4 | 55 |
| Pleckstrin | X07743 | 1 | 4.0 | 56 |
| TRAF-3 (LMP1 ass.prot.) | U19260 | 1 | 3.7 | 57 |
| hck | M16592 | 1 | 3.7 | 58 |
| IκB epsilon | M91616 | 1 | 3.5 | 59 |
| Calpain sm. Subunit | X04106 | 1 | 3.3 | 60 |
| TNFAIP3 (A20) | M59465 | 2 | 2.9 | 61 |
| IFN-regul. MxA protein | M30817 | 1 | 2.8 | 62 |
| IGFBP3 | M31159 | 1 | 2.8 | 63 |
| Lamin C | M13451 | 1 | 2.5 | 64 |
| BAT3 | M33520 | 1 | 2.3 | 65 |
| WDR1 protein | AF020056 | 1 | 2.2 | 66 |
| IRAK | L76191 | 1 | 2.1 | 67 |

TABLE 3
The effect of serum on fibronectin-induced gene induction in THP-1
cells. Differentially expressed genes are given with their GenBank
accession number and fold gene induction upon 4 hr attachment to
fibronectin versus suspension in either the presence or absence
of 2% serum. ND = Not determined.
CONFIRMED GENES

| | |
|---|---|
| GenBank | Fold Induction |

| Encoded Protein | Acc. No. | −Serum | +Serum | FNX NO |
|---|---|---|---|---|
| CRM1 | D89729 | 10.9 | | 68 |
| EST ac05e03.r1 | AA853401 | 7.4 | | 69 |
| Chr.1-linked | AL117237 | 6.7 | | 70 |
| gene | | | | |
| PAK2 | U24153 | 5.8 | | 71 |
| SGRF | AB030001 | 4.3 | | 72 |
| Glutathione | X15722 | 4.2 | | 73 |
| reductase | | | | |
| VDUP1 | S73591 | 3.1 | | 74 |
| EST yg40c04.r1 | R19741 | 2.4 | | 75 |
| Ikaros/LyF-1 | U40462 | 2.2 | | 76 |
| IF156 | X03557 | | 38.4 | 77 |
| EB1-3 | L08187 | | 30.1 | 78 |
| TNFα | X01394 | | 28.5 | 79 |
| IL-B30 | AA418955 | | 27.7 | 80 |
| MnSOD | X65965 | | 21.0 | 81 |
| MT2 pseudogene | J00272 | | 19.6 | 82 |
| CD44 | M69215 | | 18 | 83 |
| STAT5A | L41142 | | 17.0 | 84 |
| IL-8 | M28130 | | 14.5 | 85 |
| Gro-α | J03561 | | 13.8 | 86 |
| ISG15 | AH001495 | | 12.0 | 87 |
| PBEF | U02020 | | 11.1 | 88 |
| RPS20 | L06498 | | 10.5 | 89 |
| MIP-1α | D00044 | | 9.6 | 90 |
| EST ye32g07.s1 | T94880 | | 9.1 | 91 |
| LACS1 | D10040 | | 9.0 | 92 |
| SSAT | M77693 | | 8.7 | 93 |
| EST yh35f04.s1 | R26564 | | 8.5 | 94 |
| EST 42747 | AA337945 | | 8.4 | 95 |
| Ferritin heavy | L20941 | | 8.4 | 96 |
| chain | | | | |
| G0S2 | M69199 | | 7.7 | 97 |
| LITAF (PIG7) | U77396 | | 7.7 | 98 |
| Annexin VIII | X16662 | | 7.4 | 99 |
| L1 repetitive | L20941 | | 7.1 | 100 |
| sequence | | | | |
| GLCLC | M90656 | | 6.5 | 101 |
| p59 OASL | AJ225089 | | 5.9 | 102 |
| Na/K-ATPase α | M25161 | | 5.7 | 103 |
| chain | | | | |
| Fascin | U09873 | | 5.7 | 104 |
| Annexin II | M14043 | | 5.4 | 105 |
| EST q170h12.x1 | AI275149 | | 5.3 | 106 |
| sim PIM-1 | AA504311 | | 5.2 | 107 |
| sim Cyclophilin C | R32012 | | 5.2 | 108 |
| RANTES | M21121 | | 4.7 | 109 |
| IFI41 | L22342 | | 4.7 | 110 |
| EST nn79b06.s1 | AA586814 | | 4.6 | 111 |
| Adipophilin | X97324 | | 4.5 | 112 |
| CsA-binding protein | AI471997 | | 4.4 | 113 |
| Cartilage gp39 | Y08378 | | 4.4 | 114 |
| EST yg16e10.r1 | R18093 | | 4.2 | 115 |
| EST z134a01.s1 | AA131383 | | 4.1 | 116 |
| PGDH | U30255 | | 4.1 | 117 |
| Pleckstrin | X07743 | | 4.0 | 118 |
| Re1B (1-Rel) | M83221 | | 3.8 | 119 |
| hck | M16592 | | 3.7 | 120 |
| TRAF-3 | U19260 | | 3.7 | 121 |
| IκB epsilon | M91616 | | 3.5 | 122 |

FRAGMENT
DISTRIBUTION

| | |
|---|---|
| GenBank | Fold Induction |

| Encoded Protein | Acc. No. | −Serum | +Serum | Scale | FNX NO |
|---|---|---|---|---|---|
| EST seq2173 | T24017 | | 3.5 | | 123 |
| Leu-13 (9-27) | J04164 | | 3.4 | | 124 |
| Calpain sm. | X04106 | | 3.3 | 10:1 | 125 |
| Subunit | | | | | |
| Gro-β | M36820 | | 3.1 | | 126 |
| c17orf1 | AJ008112 | | 3.1 | 5:1 | 127 |
| Kynureninase | U57721 | | 3.0 | | 128 |
| TNFAIP3 (A20) | M59465 | | 2.9 | 3:1 | 129 |
| JUN-B (G0S3) | M29039 | | 2.8 | | 130 |
| IGFBP3 | M31159 | | 2.8 | 1.5:1 | 131 |
| CD82 | X53795 | | 2.8 | | 132 |
| IFN-regul.MxA | M30817 | | 2.8 | 1:1 | 133 |
| protein | | | | | |
| CD18 | M15395 | | 2.6 | | 134 |
| Lamin C | M13451 | | 2.5 | 1:1.5 | 135 |
| IL-1ra | AF043143 | | 2.4 | | 136 |
| ATF3 | H03023 | | 2.4 | 1:3 | 137 |
| Tissue Factor | M16553 | | 2.2 | | 138 |
| WDR1 protein | AF020056 | | 2.2 | 1:5 | 139 |
| IRF1 | L05072 | | 2.1 | | 140 |
| IRAK | L76191 | | 2.1 | 1:10 | 141 |
| EST zr98f03.s1 | AA215649 | | 2.1 | | 142 |
| VAMP8 | AF053233 | | −1.8 | | 143 |
| c-src | X03996 | 5.3 | 13.6 | ND | 144 |
| MCP-1 | S69738 | 4.2 | 38.5 | | 145 |
| MyD88 | U70451 | 3.8 | 4.6 | | 146 |
| TNFAIP2 (B94) | M92357 | 2.9 | 20.0 | | 147 |
| IP10 | X02530 | 2.7 | 10.1 | | 148 |
| IL-1β | M15330 | 2.6 | 7.8 | | 149 |
| EST yg61f07.r1 | R34755 | 2.3 | 6.9 | | 150 |
| KIAA0027 | D25217 | 2.2 | 3.7 | | 151 |
| p62 Ick ligand | U46751 | 2.2 | 9.9 | | 152 |
| NF-κB1 p105/p50 | M58603 | 2.0 | 7.9 | | 153 |
| IL-10 receptor | U00672 | 2.1 | 13.2 | | 154 |
| EST zk47f12.r1 | AA040335 | 2.0 | 8.8 | | 155 |
| EST yg21b04.r1 | R19475 | 2.0 | 2.6 | | 156 |
| EST zo23f04.s1 | AA134958 | 1.9 | 11.7 | | 157 |
| EST 76292 | AA365298 | 1.8 | 5.3 | | 158 |
| EST tw91d04.x1 | A1591110 | 1.8 | 4.4 | | 159 |
| KIAA0105 | D14661 | 1.7 | 11.9 | | 160 |
| CD40 | X60592 | 1.6 | 9.7 | | 161 |
| EST yh20d12.r1 | R21238 | 1.6 | 3.2 | | 162 |
| PU.1 | NM003120 | 1.6 | 2.5 | | 163 |
| CAGH3 | I08037 | 1.5 | 10.3 | | 164 |
| BCL3 | M31732 | 1.4 | 11.3 | | 165 |
| BAT3 | M33520 | 1.3 | 2.3 | | 166 |
| EST ov46b03.x1 | AI73404 | −2.4 | −2.7 | | 167 |
| Carbonic anhydrase | M77176 | −2.0 | −5.3 | | 168 |
| II | | | | | |

(FN120 = Fibronectin 120 kD fragment; LM = Laminin; COL = Collagen type I).
Genes linked to the NF-κB pathway are shown in green.
Colorimetric scale indicating fold change in gene expression is as shown in Table 3.

| | | Fold Induction | | | |
|---|---|---|---|---|---|
| | GenBank | | | | FNX |
| Encoded Protein | Acc. No. | FN120 | LM | COL | No |
| Spermidine N1-acetyltransferase | M77693 | 4.9 | 10.5 | 7.4 | 169 |
| Transcription factor ZNF15 | U22377 | 4.6 | 8.0 | 6.3 | 170 |
| Nuclear matrix protein SRM160 | AF048977 | 4.2 | 2.9 | 2.7 | 171 |
| TNFAIP3 (A20) | M59465 | 4.1 | 5.6 | 4.6 | 172 |
| IL-8 | M26383 | 4.0 | 5.4 | 4.4 | 173 |
| TIMP3 | U38955 | 4.0 | 5.0 | 3.2 | 174 |
| Transcription factor ELF1 | M82882 | 3.4 | 3.7 | 2.1 | 175 |
| p62 lck ligand | U46751 | 3.4 | 5.0 | 4.4 | 176 |
| sim Zn-finger prot. SNAH | AA465052 | 3.1 | 5.4 | 3.9 | 177 |
| BRCA2 | U43746 | 3.0 | 10.4 | 9.8 | 178 |
| H3.3 histone class C | M11353 | 3.0 | 10.4 | 9.8 | 179 |
| Beige protein homolog LYST | U67615 | 2.9 | 4.4 | 3.6 | 180 |
| sim Zn-finger protein PZF | AA421392 | 2.9 | 3.0 | 3.1 | 181 |
| Adipophilin | X97324 | 2.6 | 4.3 | 4.2 | 182 |
| EST zv78b08.r1 | AA429083 | 2.6 | 6.4 | 4.9 | 183 |
| EST zq40h11.s1 | AA160975 | 2.6 | 6.4 | 4.9 | 184 |
| PEC iota | I18964 | 2.6 | 2.7 | 4.3 | 185 |
| PECAM-1 | M28526 | 2.6 | 2.2 | 2.0 | 186 |
| Protein tyr PPase epsilon | X54134 | 2.5 | 3.1 | 4.2 | 187 |
| Palmitoyl-protein thioesterase | L42809 | 2.5 | 2.3 | 3.5 | 188 |
| C-4 methyl sterol oxidase | U60205 | 2.4 | 2.4 | 3.7 | 189 |
| EST 177650 | AA306714 | 2.4 | 2.6 | 4.1 | 190 |
| Steroid dehydrogenase KIK-1 | AA070298 | 2.3 | 2.4 | 2.5 | 191 |
| hom. | | | | | |
| Aspartyl tRNA synthetase (DARS) | J05032 | 2.3 | 2.1 | 2.4 | 192 |
| CD18 | M15395 | 2.2 | 2.3 | 2.0 | 193 |
| Transforming protein rhoA | X05026 | 2.2 | 1.7 | 1.6 | 194 |
| Groβ | M36820 | 2.2 | 3.5 | 3.6 | 195 |
| TNFAIP2 (B94) | M92357 | 2.2 | 4.7 | 3.3 | 196 |
| EST 72341 | AA362484 | 2.1 | 2.3 | 2.0 | 197 |
| Squalene Monooxygenase | D78130 | 2.1 | 4.2 | 4.4 | 198 |
| (ERG1) | | | | | |
| TANK | U63830 | 1.9 | 3.0 | 2.8 | 199 |
| Plaminogen | M74220 | 1.9 | 4.6 | 3.8 | 200 |
| Transcription Factor NRF2 | S74017 | 1.8 | 3.0 | 2.5 | 201 |
| 1, 4 α-glucan branch. | L07956 | 1.7 | 2.2 | 2.1 | 202 |
| enzyme GBE1 | | | | | |
| PBEF | U02020 | 1.6 | 3.4 | 2.2 | 203 |
| LDL receptor | L29401 | 1.6 | 2.0 | 1.7 | 204 |
| IL-1β | M15330 | 1.5 | 2.5 | 1.5 | 205 |
| 40S ribosomal protein S2 | X17206 | 1.5 | 2.3 | 1.7 | 206 |
| EST yb25f12.s1 | T51565 | −9.9 | −9.1 | −4.1 | 207 |
| Cyclin F | Z36714 | −3.2 | −5.0 | −3.6 | 208 |
| EST no14b05.s1 | AA601191 | −2.4 | 5.5 | −8.2 | 209 |

Central to immune and inflammatory responses are the integrin-mediated adhesive interactions of cells with their extracellular matrix (ECM)-rich environment. Using a comprehensive and quantitative mRNA profiling technique, we analyzed the effect of ECM-induced attachment on monocyte gene expression, its regulation by growth factors and the integrin-specificity of this event. Adhesion of cells to fibronectin resulted in increased expression of a large number of genes, which was strongly potentiated by the presence of growth factors. Adhesion activated both the NF-kB and Jak-STAT pathways of gene transcription, and increased expression of genes involved in inflammatory and immune responses, revealing the importance of ECM-integrin interactions in these processes.

Macrophages are present in almost every tissue; the tissue macrophage pool is 500-1000 times larger than the bone marrow and blood pools (1). This wide distribution within the peripheral tissues reflects the fact that these cells play an essential role in the disposal of foreign agents, in the initiation and mediation of immune and inflammatory responses, as well as in the repair process following tissue injury. Human blood monocytes undergo differentiation to macrophages upon migration into the extracellular matrix (ECM)-rich environment of extravascular tissue (2). Attachment of cells to ECM proteins, such as fibronectin, collagen and laminin, is mediated predominantly by members of the integrin family of adhesion molecules (3). Among the various ECM proteins, fibronectin plays a key role in promoting cell adhesion and various functions of monocytes and macrophages. Attachment of monocytes to fibronectin, which occurs primarily through the α4β1 and α5β1 integrin heterodimers, promotes cell migration, phagocytosis, differentiation, and modulates the secretion of inflammatory mediators (4-7). Leukocyte proliferation and cytokine secretion can be synergistically regulated by the combination of integrin-mediated adhesion and soluble growth factors, demonstrating the intimate collaboration that exists between growth factors and integrins (8).

We investigated on a global and unbiased basis, the ability of ECM-integrin interactions to regulate monocyte gene expression using a recently published restriction enzyme-based method (GeneCalling™) for identifying differentially expressed genes (9). GeneCalling couples expression profiling with a database query which utilizes fragment length and end sequence information to provide immediate feedback on expressed genes. As GeneCalling requires no a priori gene sequence, it is an open system that detects both known and novel genes. GeneCalling provides >95% coverage of the expressed genome with a sensitivity of detection greater than 1:100,000 and can detect gene expression changes down to 1.5-fold differences (9). This approach has been used to identify genes linked to cardiac hypertrophy (9), obesity (10), and hematopoietic differentiation (11).

Using this technology, we investigated the effect of ECM-induced adhesion on monocyte gene expression, its regulation by growth factors, and the integrin-specificity of this event. The human monocytic cell line, THP-l, is a well-studied model system for examining integrin-induced gene expression in circulating leukocytes (6, 12). To address the first two issues, THP-1 cells were either left in suspension or plated on fibronectin for 4 hrs in either the presence or absence 2% fetal calf serum. To examine integrin-specific gene expression, THP-1 cells were left either in suspension or plated on a variety of different ECM proteins (RGD-containing 120 kD fragment of fibronectin, laminin, collagen type I) for 1 hr in the absence of serum. Differential gene expression was quantified using GeneCalling (9, 13).

The comparisons analyzed in this study are listed in Table 1 with the total number of fragments detected and the number of differentially expressed fragments (±/−2-fold) listed. Attachment of cells for 4 hours to fibronectin under serum-containing conditions resulted in 1189 fragments (4.4%) being differentially expressed relative to suspension cells also cultured in the presence of serum (Table 1).

TABLE 1
Global analysis of monocyte gene expression changes mediated
through attachment to extracellular matrix molecules. The
indicated comparisons were made, and the magnitude of gene
expression change is reflected by both the number and
percentage of gene fragments that are differentially expressed.

| | | Number of Fragments | Number of Fragments | Change in Gene |
|---|---|---|---|---|
| | Time | Detected | Differentially Expressed | Expression |
| Type of Subtraction | (hours) | (all fragments) | (fragments +/− 2-fold) | (%) |
| TYPE I COLLAGEN VS SUSPENSION | 1 | 26 099 | 896 | 3.4 |
| LAMININ VS SUSPENSION | 1 | 26 443 | 1009 | 3.7 |
| FIBRONECTIN 120 kD VS SUSPENSION | 1 | 26 924 | 733 | 2.7 |
| FIBRONECTIN vs SUSPENSION | 4 | 22 494 | 281 | 1.2 |
| FIBRONECTIN + SERUM vs | 4 | 27 253 | 1189 | 4.4 |
| SUSPENSION + SERUM | | | | |

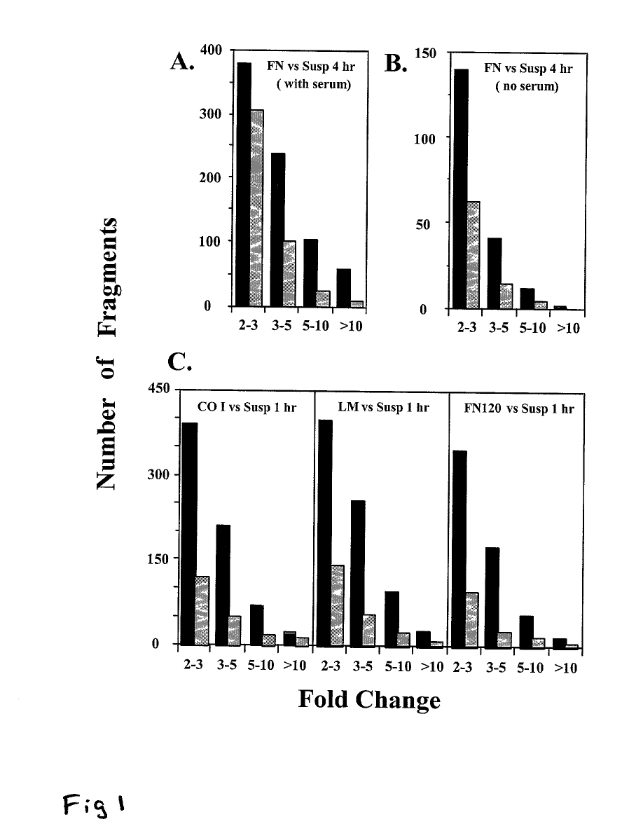

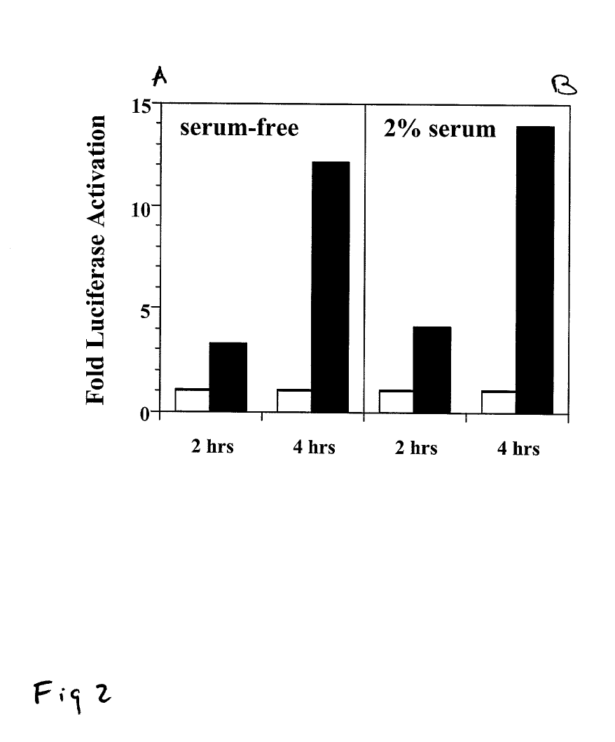

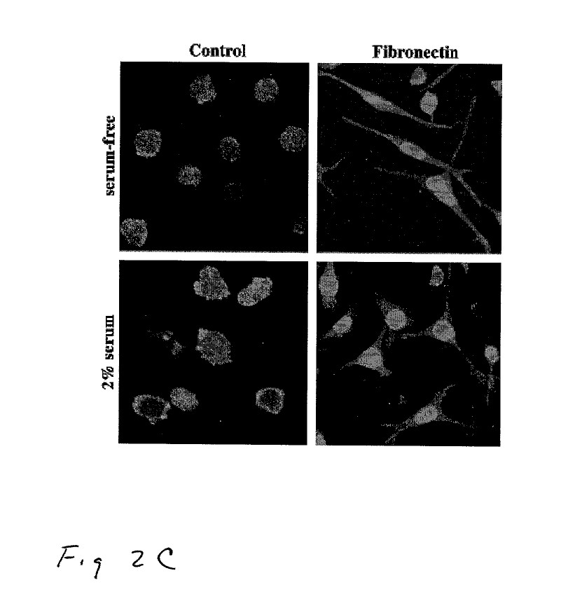

The majority of the 1189 differentially expressed fragments are upregulated upon attachment to fibronectin, including over 150 fragments whose expression is increased by >5-fold (FIG. 1 A). We positively identified the genes corresponding to 113 of the most highly induced fragments using competitive PCR. The 113 fragments represented a sixth of all upregulated fragments and segregated into 87 distinct clusters, representing 67 genes and 20 EST fragments (Tables 2 and 3). These genes and EST fragments represent a five-fold increase in the number of matrix-induced genes identified to-date (6, 12, 14, 15). The sensitivity and reproducibility of GeneCalling is highlighted by the fact that among the 67 known genes were 14 genes previously identified as being induced upon monocyte attachment to fibronectin (6, 12, 14, 15). Real-time PCR results on over a dozen genes, using independent experimental samples, confirmed in all cases the direction and magnitude of the gene expression changes observed (16).

The list of known genes that are induced by attachment to fibronectin include: secreted and cell surface molecules; metabolic proteins and intracellular regulatory proteins; and transcription factors (Table 2). 46 of the 67 identified genes induced by fibronectin attachment in the presence of serum are known to be linked to or regulated through either the NF-kB (28 genes) or Jak/STAT (18 genes) pathways of gene transcription (Table 2) (17, 18). Included among the 28 NF-kB-regulated genes are 14 genes previously identified as being induced by fibronectin attachment (A20 protein, Gro-α, Gro-β, IkB, IL-1β, IL-1ra, IL-8, JunB, MCP-1, MIP-1α, NF-kB, superoxide dismutase, tissue factor, TNFα) (6, 12, 14, 15). We have now discovered an additional 14 NF-kB-regulated genes that can be induced by fibronectin attachment (EB13, RANTES, cartilage gp39, IL-10 receptor, CD40, ferritin heavy chain, GLCLC, BCL3, ATF3, RelB, TNFAIP2, MyD88, TRAF-3, IRAK) (Table 2). The upregulation of numerous regulators of the NF-kB activation cascade upon fibronectin binding (IRAK, MyD88, p62 lck ligand) may be suggestive of a role for these proteins in integrin-mediated activation of NF-kB.

Among the genes linked to the Jak/STAT pathway, only the expression of STAT5A itself is directly regulated by adhesion to fibronectin (Table 2). Similarly, fibronectin has recently been shown to increase activity and expression of STAT5A in endothelial cells (19). In addition to STAT5A, we have now identified 17 other fibronectin-inducible genes whose expression is known to be regulated through the Jak/STAT pathway (Table 2). Among these genes are many well-known interferon-inducible and Jak/STAT-regulated genes, such as spermidine/spermine N1-acetyltransferase, Leu-13 (9-27), IFI56, IFI41, and p59 oligoadenylate synthetase (18).

Given the synergy that exists between growth factor receptor and integrin signaling, we sought to compare the pattern of ECM-induced gene expression seen in the presence of growth factors with that seen in its absence. In contrast to the 1189 fragments differentially expressed by fibronectin attachment in the presence of serum, attachment of cells for 4 hours to fibronectin under serum-free conditions resulted in only 281 fragments being differentially expressed as compared to serum-free suspension cells (Table 1). Similar to that seen previously under serum-containing conditions, attachment to fibronectin under serum-free conditions resulted in over 70% of the differentially expressed fragments being upregulated (FIG. 1 B). Comparison of the fragments differentially regulated by fibronectin under serum-free and serum-containing conditions revealed overlap between the two (Table 3). Out of the 281 fragments regulated by fibronectin attachment in the absence of serum: 79 were also regulated by fibronectin in the presence of serum, and 202 fragments were regulated by fibronectin only in the absence of serum. Synergy between ECM and growth factors was revealed by the large number of fragments (1110) whose fibronectin-specific regulation required the presence of serum. This synergy was also evident in the increased induction of genes attached to fibronectin in the presence versus the absence of serum (Table 3).

Analysis of gene expression patterns revealed that attachment of cells to fibronectin can activate genes regulated by the NF-kB transcription pathway independent of growth factors, while ECM-induced activation of the Jak/STAT-regulated genes required the presence of growth factors (Tables 3 and 4). To extend these results, luciferase reporter, nuclear translocation, and phosphorylation assays were performed on THP-1 cells under adherent and non-adherent conditions in the presence or absence of growth factors. Fibronectin attachment activated NF-kB-mediated nuclear translocation and transcription (FIG. 2 A and 2 B). Consistent with the required presence of growth factors in ECM-mediated Jak/STAT pathway activation, attachment of cells to fibronectin in the presence of serum resulted in activation of the Jak/STAT pathway as assessed by phosphorylation of STAT1; no such activation was seen upon fibronectin attachment in the absence of serum (FIG. 2 C).

TABLE 4
Effect of growth factors on integrin-mediated activation of the NF-κB
and Jak-STAT pathways of gene transcription. The names of the
differentially expressed genes are given with their GenBank accession
number and the fold gene induction upon 4 hr attachment to fibronectin
versus suspension in either the presence or absence of 2% serum.

| | Fold Induction | |
|---|---|---|
| Encoded Protein | FN (−serum) | FN (+serum) |
| NF-kB pathway | | |
| NF-κB1 p105/p50 | 2.0 | 6.0 |
| MCP-1 | 4.2 | 38.5 |
| TNFAIP2 (B94) | 2.9 | 20.0 |
| IL-1β | 2.6 | 7.8 |
| CD40 | 1.6 | 9.7 |
| Jak-Stat pathway | | |
| STAT5A | 0 | 17.0 |
| IFI56 | 0 | 38.0 |
| ISG15 | 0 | 11.8 |
| Spermine/spermidine N1-acetyltransferase | 0 | 8.7 |
| p59 oligoadenylate synthetase | 0 | 5.9 |
| PIM-1 | 0 | 5.2 |
| Leu-13 (9-27) | 0 | 3.4 |

To investigate the effect of different integrin ligands on gene expression, we adhered THIP-1 for 1 hr on plates coated with ECM proteins specific for α2β1 (type I collagen), α5β1 (RGD-containing 120kD fragment of fibronectin), and α6β1 (laminin) integrins. Inhibition of adhesion with blocking mAbs to the relevant α subunits confirmed the specifcity of the integrin-ECM interactions (data not shown). Compared to cells left in suspension, attachment of THP-1 cells to various ECM proteins results in the change of a large number of genes (700-1000 fragments representing 2.7-3.7% of the expressed genome) (Table 1)

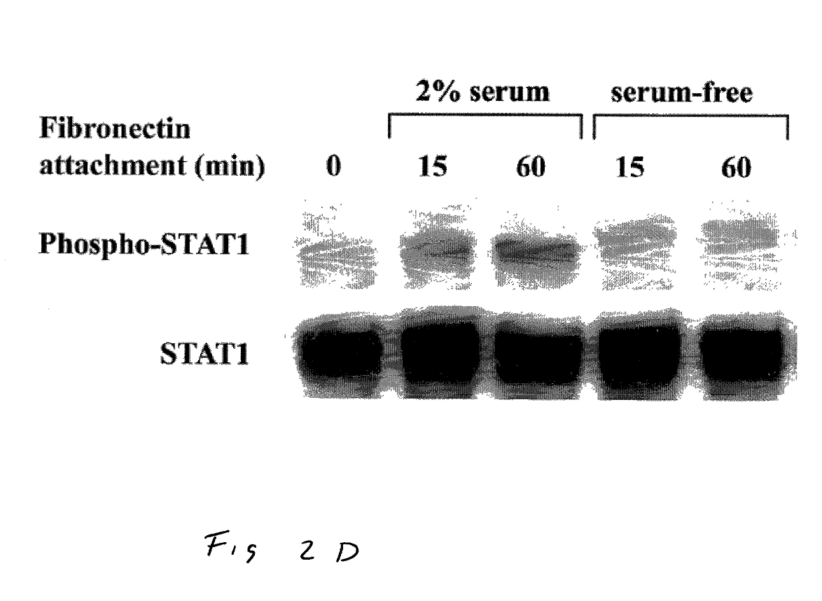

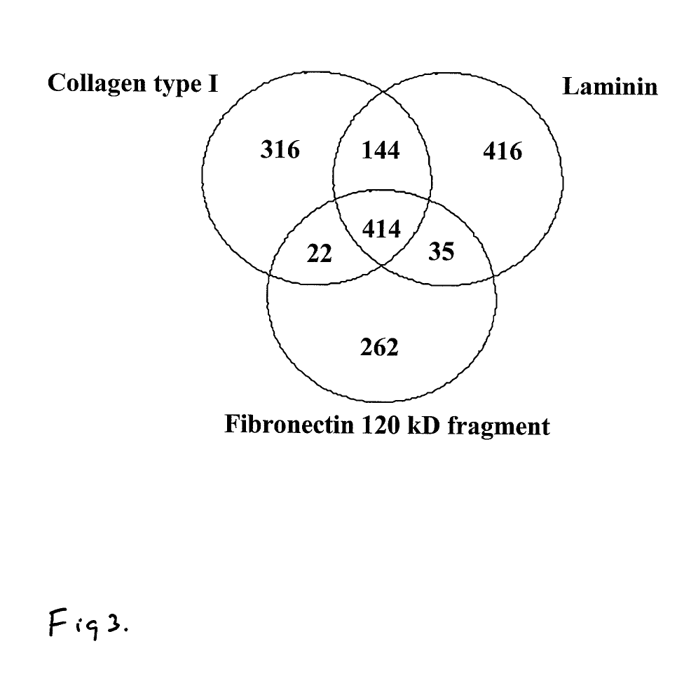

In all cases, integrin engagement resulted in roughly 80% of differentially expressed fragments showing an increase in mRNA expression (FIG. 1 C). To investigate the importance of integrin-specificity in regulating gene expression, we compared the distribution of differentially regulated fragments induced by specific integrin-ECM interactions (FIG. 3). Among the differentially expressed fragments, roughly half (414 fragments) are common to all three matrix molecules. A strong, degree of integrin specificity exists, as most of the rest of the differentially expressed fragments are regulated only through specific integrin-ECM interactions (collagen, 316 fragments; laminin, 416 fragments; 120 kD fibronectin, 262 fragments) (FIG. 3). The one notable exception to this is the 144 differentially expressed fragments that are shared between collagen type I and laminin, but are not regulated by attachment to the 120 kD fragment of fibronectin. Gene identification both confirmed genes that had previously been shown to be inducible through attachment to multiple extracellular matrices (IL-1β, IL-8, Groβ, A20 protein) (12), and resulted in the discovery of another 37 such genes (Table 5). Many of the genes commonly regulated through engagement of α2β1, α5β1 and α6β1 integrins are genes whose expression is induced through activation of NF-kB (Table 5).

Global gene expression analysis has enabled us to quantitate for the first time the relative contribution of integrin-mediated cell attachment to changes in gene expression. Our results demonstrate the effect integrin engagement has on gene expression and highlights the ability of growth factors and different matrix molecules to influence integrin-mediated gene expression. The positive identification of over 140 genes and ESTs whose expression in monocytes is induced by attachment to ECM not only represents a significant increase in the number of genes previously identified, but also reveals the importance of ECM-integrin interaction in multiple aspects of monocyte biology.

Monocyte extravasation and attachment in ECM-rich tissues that occurs during the course of immune and inflammatory responses, and mimicked here by attachment of cells to ,fibronectin, revealed many genes important in monocyte migration and activation. Monocyte attachment to the ECM results in the upregulation of numerous chemokines (IL-8, Gro-α, Gro-β, MCP-1, MIP-1α, RANTES, IP10) and cell adhesion molecules (CD18, CD44) important in attracting other cells to inflammatory sites (20, 21). Genes important in monocyte activation and effector function are also upregulated by fibronectin attachment, and include secreted molecules (IL-1β, TNFα) and cell surface molecules (CD40, CD44) (22, 23). Fibronectin attachment can also affect macrophage activation and function through increased expression of intracellular molecules (hck, MyD88) that are directly involved in macrophage activation and cytokine production (24, 25). Matrix-induced attachment also changes expression of genes (pleckstrin, calpain) that, through alterations to the cytoskeleton, are important in the regulation of monocyte cell spreading, migration, and phagocytosis (26, 27).

Along with induction of numerous genes that increase the cytolytic and cytotoxic effect of macrophages, matrix-induced attachment also results in the induction of genes responsible for protecting the cells from self-lysis. NF-kB activation initiates a response that blunts the apoptotic action of several agonists of cell death (28, 29). Among the genes induced by fibronectin attachment are several NF-kB regulated genes (MnSOD, A20, Bcl-3) that have been shown to have anti-apoptotic function (30-32). Other matrix-regulated genes not linked to NF-kB activation may also play a role in preventing apoptosis. For instance, enforced expression of PIM-1 kinase enhances growth factor-independent survival and inhibits apoptosis in murine myeloid cells (33).

Attachment of monocytes to ECM has been demonstrated to induce differentiation to macrophages (4, 7). We found that attachment to fibronectin resulted in the upregulated expression of an array of genes previously associated with differentiation of monocytes to macrophages including: CD44, ferritin, spermidine/spermine acetyltransferase (SSAT), cartilage gp39, and CD82 (34, 35). Fibronectin-mediated attachment also resulted in increased expression of several key transcriptional regulators of macrophage differentiation, including PU.1, STAT5A, and IRF-1 (36-38).

Identification of matrix-induced genes may also help in understanding the role of monocytes in disease. Both attachment to extracellular matrix and monocyte activation/differentiation are hypothesized to play an important role in the development of atherosclerosis (39). Atherosclerotic plaque formation is characterized by the accumulation of lipid droplets in macrophage-derived foam cells. Fatty acid synthesis requires a source of acetyl CoA and NADPH, and among the genes induced by attachment to fibronectin are several enzymes (phosphogluconate, kynureninase, LACS1) that promote production both acetyl CoA and NADPH (40, 41). In addition, LACS1 is also a key protein that promotes intracellular fatty acid retention (42). Consistent with this hypothesis is the finding that attachment of peripheral blood monocytes to a collagen matrix not only induces cellular differentiation but also increases intracellular lipid accumulation (7). Lastly, expression of matrix-regulated genes, such as ferritin and FNXphilin, were found to be induced in macrophages in early human atherosclerotic plaques, and their high level of expression is associated with the development of plaques and disease progression (43, 44). Thus, monocyte adherence to ECM proteins, such as fibronectin and collagen, may contribute to the development of atherosclerosis not only through increased expression of numerous cytokines and chemokines already implicated in the development of atherosclerosis, but also through increased expression of numerous metabolic enzymes and proteins that play a key role in lipid metabolism.

The identification of adherence-induced genes allowed us to further dissect integrin-mediated signal transduction pathways in monocytes. Using a global approach, we have demonstrated that the majority of matrix-induced genes are ones known to be under the control of the NF-kB and Jak/STAT transcriptional pathways. We have confirmed that ECM-induced attachment can result in activation of NF-kB (6, 12), and have demonstrated for the first time that the presence of growth factors can synergistically increase integrin-mediated activation of the NF-kB pathway and lead to the activation of the Jak/STAT pathway. Comparison of cell attachment to different ECM molecules allowed us to also investigate the relative importance of integrin specificity in affecting gene expression. The ability of different integrin-ECM interactions to regulate a large number of the same genes is not surprising, given that changes in gene expression are not the consequence of specific integrin engagement alone, but may also require adhesion, cell shape change, and cytoskeletal rearrangement (6). While the finding that distinct integrin engagement can result in different patterns of gene expression has been documented (45), we have determined for the first time, using an unbiased global approach, that the degree of integrin-specific gene expression is considerable.

Identification of genes whose expression is increased by interaction with ECM reveals an important role for ECM-integrin interactions in affecting monocyte function and thus impacting on the development of pathologies, such as atherosclerosis. This is of particular relevance in the context of inflammation and the localization of both resident and infiltrating monocytes at an inflammatory site. The importance of integrin interaction with the ECM-rich environment of peripheral tissues was recently demonstrated in numerous in vivo models of inflammation, including asthma, hypersensitivity, and arthritis (46, 47).

Screening for Integrin Modulating Agents

In one aspect, the invention provides a method of identifying integrin modulating agents. By integrin modulating agent is meant that the agent modulates (i.e., increases or decreases) integrin levels or activity. These agents include for example, integrin activators, inhibitors, endogenous and exogenous ligands, and integrin-binding proteins. Integrin ligands include for example extracellular matrix proteins, such fibronectin, collagen and laminin, and other cell surface proteins. Integrin inhibitors include small molecules, e.g. cyclic peptides; peptidomimetics; antibodies, e.g. LM609, an alphavbeta3-disrupting antibody; and tight-binding inhibitors, eg. BIO-1211, which inhibits integrin-mediated inflammation. Integrin-binding proteins include for example intracellular, extracellular and plasma membrane-associated proteins involved in cell shape, motility, proliferation, differentiation, and adhesion.

The integrin modulating agent can be identified by providing a cell population that includes cells capable of expressing one or more nucleic acid sequences homologous to those listed in Tables 2,3 and 5 as FNX 1-209. Preferably, the cell population includes cells capable of expressing one or more nucleic acids sequences homologous to FNX 1-209. By “capable of expressing” is meant that the gene is present in an intact form in the cell and can be expressed. The sequences need not be identical to sequences including FNX 1-209, as long as the sequence is sufficiently similar that specific hybridization can be detected. Preferably, the cell includes sequences that are identical, or nearly identical to those identifying the FNX nucleic acids shown in Tables 2,3 and 5.

Expression of one, some, or all of the FNX sequences is then detected, if present, and, preferably, measured. Using sequence information provided by the database entries for the known sequences, or the sequence information for the newly described sequences, expression of the FNX sequences can be detected (if present) and measured using techniques well known to one of ordinary skill in the art. For example, sequences within the sequence database entries corresponding to FNX sequences, or within the sequences disclosed herein, can be used to construct probes for detecting FNX RNA sequences in, e.g., northern blot hybridization analyses or methods which specifically, and, preferably, quantitatively amplify specific nucleic acid sequences. As another example, the sequences can be used to construct primers for specifically amplifying the FNX sequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

Expression can be also measured at the protein level, i.e., by measuring the levels of polypeptides encoded by the gene products described herein. Such methods are well known in the art and include, e.g., immunoassays based on antibodies to proteins encoded by the genes.

Expression level of one or more of the FNX sequences in the test cell population is then compared to expression levels of the sequences in one or more cells from a reference cell population. Expression of sequences in test and control populations of cells can be compared using any art-recognized method for comparing expression of nucleic acid sequences. For example, expression can be compared using GENECALLING® methods as described in U.S. Pat. No. 5,871,697 and in Shimkets et al., Nat. Biotechnol. 17:798-803.

In various embodiments, the expression of 2, 3, 4, 5, 6, 7, 8, 9, 10, 15, 20, 25, 35, 40, 50, 100, 150, 200 or all of the sequences represented by FNX 1-209 are measured. If desired, expression of these sequences can be measured along with other sequences whose expression is known to be altered according to one of the herein described parameters or conditions.

The FNX nucleic acids and encoded polypeptides can be identified using the information provide above. In some embodiments, the FNX nucleic acids and polypeptide correspond to nucleic acids or polypeptides which include the various sequences disclosed for each FNX polypeptide.

Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent, such as fibronectin, laminin or Type I collagen.

An alteration (i.e., increase or decrease) in expression of the nucleic acid sequence in the test cell population compared to the expression of the nucleic acid sequence in the reference cell population that has not been exposed to the test agent indicates the test agent is a integrin modulating agent.

The reference cell population includes one or more cells for which the compared parameter is known. The compared parameter can be, e.g., integrin modulating agent expression status. By “integrin modulating agent expression status” is meant that it is known whether the reference cell has had contact with an integrin modulating agent. Example of an integrin modulating agent are extracellular matrix component such as fibronectin, laminin or collagen. Whether or not comparison of the gene expression profile in the test cell population to the reference cell population reveals the presence, or degree, of the measured parameter depends on the composition of the reference cell population. For example, if the reference cell population is composed of cells that have not been treated with a known integrin modulating agent, a similar gene expression level in the test cell population and a reference cell population indicates the test agent is not an integrin modulating agent. Conversely, if the reference cell population is made up of cells that have been treated with an integrin modulating agent, a similar gene expression profile between the test cell population and the reference cell population indicates the test agent is an integrin modulating agent.

In various embodiments, a FNX sequence in a test cell population is considered comparable in expression level to the expression level of the FNX sequence if its expression level varies within a factor of 2.0, 1.5, or 1.0 fold to the level of the FNX transcript in the reference cell population. In various embodiments, a FNX sequence in a test cell population can be considered altered in levels of expression if its expression level varies from the reference cell population by more than 1.0, 1.5, 2.0 or more fold from the expression level of the corresponding FNX sequence in the reference cell population.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations.

In some embodiments, the test cell population is compared to multiple reference cell populations. Each of the multiple reference populations may differ in the known parameter. Thus, a test cell population may be compared to a first reference cell population known to have been exposed to an integrin modulating agent, as well as a second reference population known to have not been exposed to an integrin modulating agent.

The test cell population that is exposed to, i.e., contacted with, the test integrin modulating agent can be any number of cells, i.e., one or more cells, and can be provided in vitro, in vivo, or ex vivo.

In other embodiments, the test cell population can be divided into two or more subpopulations. The subpopulations can be created by dividing the first population of cells to create as identical a subpopulation as possible. This will be suitable, in, for example, in vitro or ex vivo screening methods. In some embodiments, various sub populations can be exposed to a control agent, and/or a test agent, multiple test agents, or. e.g., varying dosages of one or multiple test agents administered together, or in various combinations.

Preferably, cells in the reference cell population are derived from a tissue type as similar as possible to test cell, e.g., blood cell, immune cell or monocyte. In some embodiments, the control cell is derived from the same subject as the test cell, e.g., from a region proximal to the region of origin of the test cell. In other embodiments, the reference cell population is derived from a plurality of cells. For example, the reference cell population can be a database of expression patterns from previously tested cells for which one of the herein-described parameters or conditions (integrin modulating agent expression status is known).

The test agent can be a compound or composition (e.g., protein, nucleic acid, small molecule, or antibody) not previously described or can be a previously known compound but which is not known to be an integrin modulating agent.

The invention also includes an integrin modulating agent identified according to this screening method, and a pharmaceutical composition which includes the integrin modulating agent.

The subject is preferably a mammal. The mammal can be, e.g., a human, non-human primate, mouse, rat, dog, cat, horse, or cow.

### METHODS OF TREATING DISORDERS IN A SUBJECT

Also included in the invention is a method of treating, i.e, preventing or delaying the onset of various atherosclerosis or inflammatory disorders, e.g., asthma, hypersensitivity or arthritis a subject by administering to the subject an agent which modulates the expression or activity of one or more nucleic acids selected from the group consisting of FNX 1-209.“Modulates” is meant to include increase or decrease expression or activity of the FNX nucleic acids. Preferably, modulation results in alteration alter the expression or activity of the FNX genes or gene products in a subject to a level similar or identical to a subject not suffering from the disorder. The subject can be, e.g., a human, a rodent such as a mouse or rat, or a dog or cat.

An agent can be e.g., (i) a FNX polypeptide; (ii) a nucleic acid encoding a FNX polypeptide; (iii) a nucleic acid that increases expression of a nucleic acid that encodes a FNX polypeptide and, and derivatives, fragments, analogs and homologs thereof, (iv) and FNX antibody or (v) an a FNX antisence nucleic acid. A nucleic acid that increase expression of a nucleic acid that encodes a FNX polypeptide includes, e.g., promoters, enhancers. The nucleic acid can be either endogenous or exogenous.

The herein described FNX nucleic acids, polypeptides, antibodies, agonists, and antagonists when used therapeutically are referred to herein as “Therapeutics”. Methods of administration of Therapeutics include, but are not limited to, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g. oral mucosa, rectal and intestinal mucosa, etc.) and may be administered together with other biologically-active agents. Administration can be systemic or local. In addition, it may be advantageous to administer the Therapeutic into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

Various delivery systems are known and can be used to administer a Therapeutic of the present invention including, e.g.: (i) encapsulation in liposomes, microparticles, microcapsules; (ii) recombinant cells capable of expressing the Therapeutic; (iii) receptor-mediated endocytosis (See, e.g., Wu and Wu, 1987. J Biol Chem 262:4429-4432); (iv) construction of a Therapeutic nucleic acid as part of a retroviral or other vector, and the like. In one embodiment of the present invention, the Therapeutic may be delivered in a vesicle, in particular a liposome. In a liposome, the protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028; and 4,737,323, all of which are incorporated herein by reference. In yet another embodiment, the Therapeutic can be delivered in a controlled release system including, e.g.: a delivery pump (See, e.g., Saudek, et al., 1989. New Engl J Med 321:574 and a semi-permeable polymeric material (See, e.g., Howard, et al., 1989. J Neurosurg 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, Fla.).

In a specific embodiment of the present invention, where the Therapeutic is a nucleic acid encoding a protein, the Therapeutic nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, eg., Joliot, et al., 1991. Proc Natl Acad Sci USA 88:1864-1868), and the like. Alternatively, a nucleic acid Therapeutic can be introduced intracellularly and incorporated within host cell DNA for expression, by homologous recombination.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the Therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. However, suitable dosage ranges for intravenous administration of the Therapeutics of the present invention are generally about 20-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Polynucleotides of the present invention can also be used for gene therapy. Gene therapy refers to therapy that is performed by the administration of a specific nucleic acid to a subject. Delivery of the Therapeutic nucleic acid into a mammalian subject may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. Polynucleotides of the invention may also be administered by other known methods for introduction of nucleic acid into a cell or organism (including, without limitation, in the form of viral vectors or naked DNA). Any of the methodologies relating to gene therapy available within the art may be used in the practice of the present invention. See e.g. Goldspiel, et al., 1993. Clin Pharm 12:488-505.

Cells may also be cultured ex vivo in the presence of therapeutic agents or proteins of the present invention in order to proliferate or to produce a desired effect on or activity in such cells. Treated cells can then be introduced in vivo for therapeutic purposes.

### METHODS OF INHIBITING MONOCYTE DIFFERENTIATION

Also included in the invention is a method monocyte differentiation by contacting a cell, e.g., an agent which modulates the expression or activity of one or more nucleic acids selected from the group consisting of FNX 1-209. “Modulates” is meant to include increase or decrease expression or activity of the FNX nucleic acids.

By monocyte differentiation is meant to include the maturation of a monocyte into a macrophage. Differentiation may be measured for example by detecting cell surface markers associated with monocytes and macrophages.

An agent can be e.g., (i) a FNX polypeptide; (ii) a nucleic acid encoding a FNX polypeptide; (iii) a nucleic acid that increases expression of a nucleic acid that encodes a FNX polypeptide and, and derivatives, fragments, analogs and homologs thereof, (iv) and FNX antibody or (v) an a FNX antisence nucleic acid. A nucleic acid that increase expression of a nucleic acid that encodes a FNX polypeptide includes, e.g., promoters, enhancers. The nucleic acid can be either endogenous or exogenous.

### ANTISENSE

Another aspect of the invention pertains to isolated antisense nucleic acid molecules that are hybridizable to or complementary to the nucleic acid molecule comprising the nucleotide sequence of a FNX sequence or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire FNX coding strand, or to only a portion thereof. Nucleic acid molecules encoding fragments, homologs, derivatives and analogs of a FNX protein, or antisense nucleic acids complementary to a nucleic acid comprising a FNX nucleic acid sequence are additionally provided.

In one embodiment, an antisense nucleic acid molecule is antisense to a “coding region” of the coding strand of a nucleotide sequence encoding FNX. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding FNX. The term “noncoding region” refers to 5′ and 3′ sequences which flank the coding region that are not translated into amino acids (i.e., also referred to as 5′ and 3′ untranslated regions).

Given the coding strand sequences encoding FNX disclosed herein, antisense nucleic acids of the invention can be designed according to the rules of Watson and Crick or Hoogsteen base pairing. The antisense nucleic acid molecule can be complementary to the entire coding region of FNX mRNA, but more preferably is an oligonucleotide that is antisense to only a portion of the coding or noncoding region of FNX mRNA. For example, the antisense oligonucleotide can be complementary to the region surrounding the translation start site of FNX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylam inomethyluracil, dihydrouracil, beta-D-galactosylqueosi ne, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuraci 1, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a FNX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An o-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue el al. (1987) Nucleic Acids Res 15: 6131-6148) or a chimeric RNA -DNA analogue (Inoue et al. (1987) FEBS Lett 215: 327-330).

### RIBOZYMES AND PNA MOIETIES

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave FNX mRNA transcripts to thereby inhibit translation of FNX mRNA. A ribozyme having specificity for a FNX-encoding nucleic acid can be designed based upon the nucleotide sequence of a FNX DNA disclosed herein. For example, a derivative of a Tetrahymena L-19 IVS RNA can be constructed in which the nucleotide sequence of the active site is complementary to the nucleotide sequence to be cleaved in a FNX-encoding mRNA. See, e.g., Cech et al. U.S. Pat. No. 4,987,071; and Cech et al. U.S. Pat. No. 5,116,742. Alternatively, FNX mRNA can be used to select a catalytic RNA having a specific ribonuclease activity from a pool of RNA molecules. See, e.g., Bartel et al., (1993) Science 261:1411-1418.

Alternatively, FNX gene expression can be inhibited by targeting nucleotide sequences complementary to the regulatory region of a FNX nucleic acid (e.g., the FNX promoter and/or enhancers) to form triple helical structures that prevent transcription of the FNX gene in target cells. See generally, Helene. (1991) Anticancer Drug Des. 6:569-84; Helene. et al. (1992) Ann. N.Y. Acad. Sci. 660:27-36; and Maher (1992) Bioassays 14: 807-15.

In various embodiments, the nucleic acids of FNX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of FNX can be used in therapeutic and diagnostic applications. For example, PNAs can be used as antisense or antigene agents for sequence-specific modulation of gene expression by, e.g., inducing transcription or translation arrest or inhibiting replication. PNAs of FNX can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of FNX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of FNX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res l7: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652, PCT Publication No. W088/09810) or the blood-brain barrier (see, e.g., PCT Publication No. W089/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

### REFERENCES

1. Z. A. Cohn, Harvey Lect., 77, 63 (1983).

2. Z. Werb, in Basic and Clinical Immunology, D. P. Sites, J. P. Stobe, J. V. Wells, Eds. (Appleton-Century-Crofts, Norwalk, Conn., 1987), p. 96.

3. M. E. Hemler, Annu. Rev. Immunol., 8, 365 (1990).

4. G. Kaplan, G. Gaudernack, J. Exp. Med., 156, 1101 (1982).

5. B. Thorens, J. Mermod, P. Vassalli, Cell, 48, 671 (1987).

6. R. L. Juliano, S. Haskill, J. Cell Biol., 120, 577 (1993).

7. R. B. Wesley, X. Meng, D. Godin, Z. S. Galis, Arterioscler. Thromh. Vasc. Biol., 18, 432 (1998).

8. M. A. Schwartz, M. D. Schaller, M. H. Ginsberg, Annu. Rev. Cell Dev. Biol., 11, 549 (1995).

9. R. A. Shimkets et al, Nature Biotech., 17, 798 (1999).

10. M. Renz et al., J. Biol. Chem., 275, 10429 (2000).

11. Ohneda et al., Immunity, 12, 141 (2000).

12. J. E. Meredith et al., Endocrine Reviews, 17, 207 (1996).

13. Following double-stranded cDNA synthesis of polyA+RNA, cDNA was separated into 96 different pools and each digested with a different pair of restriction enzymes. The resulting fragments were ligated with complementary adapters, one labeled with biotin and the other with fluorescamine (FAM), and amplified by PCR. After affinity purification on streptavidin and separation by polyacrylamide gel electrophoresis, the FAM-labeled fragments were detected by laser excitation. A composite restriction fragment profile is generated for each sample, based on average peak height and variance of nine separate restriction enzyme digestions (each sample triplicate is subjected to 3 separate restriction enzyme digestions). The restriction fragment profiles of two samples are compared and differentially expressed fragments are identified. Linkage of a differentially expressed cDNA fragment to a gene is made through knowledge of the restriction enzymes used to generate the fragment ends and the length of the fragment itself. A species-specific query of databases reveals genes that fit these criteria, with gene identification confirmed by competitive PCR using gene-specific oligonucleotides.

14. S. Fan, N. Mackman, M. Cui, T. S. Edgington, J. Immunol., 154, 3266 (1995).

15. F. Marra et al., FEBS Lett., 414, 221 (1997).

16. Genes confirmed using real-time PCR: MCP-1, MnSOD, IL-8, Gro-α, MIP-1α, IP10, CD44, IL-10 receptor, CD40, glutamate-cysteine ligase catalytic subunit, NF-kB1, TNFAIP2, and p62 lck ligand.

17. P. A. Baeuerle, T. Henkel, Annu. Rev. Immunol., 12, 141 (1994).

18. J. E. Darnell, Science, 277, 1630 (1997).

19. M. F. Brizzi et al., Mol. Biol. Cell, 10, 3463 (1999).

20. M. Baggiolini, B. Dewald, B. Moser, Annu. Rev. Immunol., 15, 675 (1997).

21. T. A. Springer, Cell, 76, 301 (1994).

22. I. S. Grewal, R. A. Flavell, Annu. Rev. Immunol., 16, 11 (1998).

23. D. S. Webb, Y. Shimuzi, G. A. van Seventer, S. Shaw, T. L. Gerrard, Science, 249, 1295 (1990).

24. B. K. English, J. N. Ihle, A. Myracle, T. Yi, J. Exp. Med., 178, 1017 (1993).

25. T. Kawai, O. Adachi, T. Ogawa, K. Takeda, S. Akira, Immunity, 11, 115 (1999).

26. J. H. Brumell et al., J. Immunol., 163, 3388 (1999).

27. S. Kulkarni, T. C. Saido, K. Suzuki, J. E. B. Fox, J. Biol. Chem., 274, 21265 (1999).

28. A. A. Beg, D. Baltimore, Science, 274, 782 (1996).

29. D. J. Van Antwerp, S. J. Martin, T. Kafri, D. R. Green, l. M. Verma, Science, 274, 787 (1996).

30. Fridovich, J. Biol. Chem., 264, 7761 (1989).

31. A. W. Opipari, H. M. Hu, R. Yabkowitz, V. M. Dixit, J. Biol. Chem., 267, 12424 (1992).

32. A. Rebollo et al., Mol. Cell. Biol., 20, 3407 (2000).

33. M. Lilly, A. Kraft, Cancer Res., 57, 5348 (1997).

34. S. W. Krause et al., J. Leukoc. Biol., 60,540 (1996).

35. M. C. Gingras, J. F. Margolin, Exp. Hematol., 28, 65 (2000).

36. A. F. Valledor, F. E. Borras, M. Cullell-Young, A. Celada, J. Leukoc. Biol., 63, 405 (1998).

37. M. Kieslinger et al., Genes & Development, 14, 232 (2000).

38. J. Xaus et al., Immunity, 11, 103 (1999).

39. R. Ross, N. Engl. J. Med., 340, 115 (1999).

40. F. B. Hillgartner, L. M. Salati, A. G. Goodridge, Physiol. Rev., 75, 47 (1995).

41. M. P. Heyes, C. Y. Chen, E. O. Major, K. Saito, Biochem. J., 326, 351 (1997).

42. J. E. Schaffer, H. F. Lodish, Cell, 79, 427 (1994).

43. J. H. Pang et al., J. Clin. Invest., 97, 2204 (1996).

44. X. Wang et al., FEBS Lett., 462, 145 (1999).

45. P. Huhtala et al., J. Cell Biol., 129, 867 (1995).

46. W. R. Henderson et al., J. Clin. Invest., 100, 3083 (1997).

47. A. R. de Fougerolles et al., J. Clin. Invest., 105, 721 (2000).

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of modulating the expression of at least one gene in a monocyte cell wherein expression of said gene is responsive to adhesion of said monocyte cell to extracellular matrix or a component thereof, said method comprising contacting said monocyte with an agent that modulates the expression of said gene.

2. The method of claim 1, wherein the component of extracellular matrix is fibronectin.

3. The method described of claim 1, wherein the gene is chosen from the group consisting of a gene encoding a secreted factor induced in a monocyte in response to adhesion of to extracellular matrix or a component thereof, a gene encoding a cell surface protein induced in a monocyte in response to adhesion of to extracellular matrix or a component thereof, a gene encoding a metabolic protein induced in a monocyte in response to adhesion of to extracellular matrix or a component thereof, a gene encoding a transcription factor induced in a monocyte in response to adhesion of to extracellular matrix or a component thereof, and a gene encoding a regulatory protein induced in a monocyte in response to adhesion of to extracellular matrix or a component thereof.

4. The method of claim 1, wherein the gene comprises a nucleotide sequence provided by a GenBank Accession Number chosen from the group consisting of S69738, L08187, X01394, AA418955, M28130, J03561, AH001495, U02020, X02530, D00044, M15330, M21121, Y08378, M36820, AF043143, M69215, U00672, X60592, J04164, X53795, M155395, M16553, X65965, L06498, D10040, M77693 , L20941, M90656,, X97324, U30255, U57721, L41142, M31732, Z47744, U77396, M83221, M29039, NM003120, H03023, L05072, X03557, M92357, X03996, U46751, M69199, X16662, AJ225089, M25161, U09873, M14043, R32012, AA50431 1, L22342, U70451, A1471997, X07743, U19260, M16592, M91616, X04106, M59465, M30817, M31159, M13451, M33520, AF020056, and L76191.

5. The method of claim 3, wherein the gene encodes a secreted factor induced in a monocyte in response to adhesion of to extracellular matrix or a component thereof, said gene comprising a nucleotide sequence provided by a GenBank Accession Number chosen from the group consisting of S69738, L08187, X01394, AA418955, M28130, J03561, AH001495, U02020, X02530, D00044, M15330, M21121, Y08378, M36820, and AF043143.

6. The method described of claim 3, wherein the gene encodes a cell surface protein induced in a monocyte in response to adhesion of to extracellular matrix or a component thereof, said gene comprising a nucleotide sequence provided by a GenBank Accession Number chosen from the group consisting of M69215, U00672, X60592, J04164, X53795, M15395, and M16553.

7. The method of claim 3, wherein the gene encodes a metabolic protein induced in a monocyte in response to adhesion of to extracellular matrix or a component thereof, said gene comprising a nucleotide sequence provided by a GenBank Accession Number chosen from the group consisting of X65965, L06498, D10040, M77693 , L20941, M90656, X97324, U30255, and U57721.

8. The method of claim 3, wherein the gene encodes a transcription factor induced in a monocyte in response to adhesion of to extracellular matrix or a component thereof, said gene comprising a nucleotide sequence provided by a GenBank Accession Number chosen from the group consisting of L41142, M31732, Z47744, U77396, M83221, M29039, NM003120, H03023, and L05072.

9. The method of claim 3, wherein the gene encodes a regulatory protein induced in a monocyte in response to adhesion of to extracellular matrix or a component thereof. said gene comprising a nucleotide sequence provided by a GenBank Accession Number chosen from the group consisting of X03557, M92357, X03996, U46751, M69199, X16662, AJ225089, M25161, U09873, M14043, R32012, AA504311, L22342, U70451, AI471997, X07743, U19260, M16592, M91616, X04106, M59465, M30817, M31159, M13451, M33520, AF020056, and L76191.

10. The method of claim 4, wherein the gene comprises a nucleotide sequence provided by a GenBank Accession Number chosen from the group consisting of S69738, L08187, X01394, AA418955, M28130, J03561, AH001495, U02020, X02530, D00044, M15330, M69215, U00672, X60592, X65965, L06498, D10040, M77693 , L20941, M90656, L41142, M31732, Z47744, U77396, X03557, M92357, X03996, U46751, M69199, X16662, AJ225089, M25161, U09873, M14043, R32012, and AA50431 1.

11. The method of claim 1, wherein the agent hybridizes to at least a portion of the nucleic acid comprising the gene.

12. A method of inhibiting differentiation of a monocyte to a macrophage comprising contacting said monocyte with an agent that modulates the expression of at least one gene in said monocyte cell wherein expression of said gene is responsive to adhesion of said monocyte cell to extracellular matrix or a component thereof, said modulation being effective to inhibit the differentiation of the monocyte.

13. A method of interfering with development of atherosclerotic plaque wherein development of atherosclerotic plaque is associated with activation of a monocyte upon contact with extracellular matrix or a component thereof, said method comprising contacting said monocyte with an agent that modulates the expression of at least one gene in said monocyte cell wherein expression of said gene is responsive to adhesion of said monocyte cell to extracellular matrix or a component thereof, said modulation being effective to inhibit the activation of the monocyte.

14. A method of interfering with development of an inflammatory condition wherein development of the inflammatory condition is associated with differentiation of a monocyte upon contact with extracellular matrix or a component thereof, said method comprising contacting said monocyte with an agent that modulates the expression of at least one gene in said monocyte cell wherein expression of said gene is responsive to adhesion of said monocyte cell to extracellular matrix or a component thereof, said modulation being effective to inhibit the differentiation of the monocyte.

15. The method of claim 14, wherein the inflammatory condition is chosen from the group consisting of asthma, hypersensitivity and arthritis.

16. The method of claims 12, 13, or 14, wherein the monocyte is contacted with an antibody to a protein encoded by the nucleic acid sequence of a gene chosen from the group consisting of a gene encoding a secreted factor induced in a monocyte in response to adhesion of to extracellular matrix or a component thereof, and a gene encoding a cell surface protein induced in a monocyte in response to adhesion of to extracellular matrix or a component thereof.

17. The method of claim 16 wherein the gene is a gene encoding a secreted factor induced in a monocyte in response to adhesion of to extracellular matrix or a component thereof, said gene comprising a nucleotide sequence provided by a GenBank Accession Number chosen from the group consisting of S69738, L08187, X01394, AA418955, M28130, J03561, AH001495, U02020, X02530, D00044, M1 5330, M21121, Y08378, M36820, and AF043143.

18. The method of claim 16 wherein the gene is a gene encoding a cell surface protein induced in a monocyte in response to adhesion of to extracellular matrix or a component thereof, said gene comprising a nucleotide sequence provided by a GenBank Accession Number chosen from the group consisting of M69215, U00672, X60592, J04164, X53795, M15395, and M16553.

19. The method of claim 1, 12, 13, or 14, wherein the monocyte is contacted with an antibody to a protein encoded by the nucleic acid sequence of a gene chosen from the group consisting of a gene encoding a secreted factor induced in a monocyte in response to adhesion of to extracellular matrix or a component thereof, and a gene encoding a cell surface protein induced in a monocyte in response to adhesion of to extracellular matrix or a component thereof.

20. The method of claim 16, wherein the gene is a gene encoding a secreted factor induced in a monocyte in response to adhesion of to extracellular matrix or a component thereof, said gene comprising a nucleotide sequence provided by a GenBank Accession Number chosen from the group consisting of S69738, L08187, X01394, AA418955, M28130, J03561, AH001495, U02020, X02530, D00044, M15330, M21121, Y08378, M36820, and AF043143.

21. The method of claim 16, wherein the gene is a gene encoding a cell surface protein induced in a monocyte in response to adhesion of to extracellular matrix or a component thereof, said gene comprising a nucleotide sequence provided by a GenBank Accession Number chosen from the group consisting of M69215, U00672, X60592, J04164, X53795, M15395, and M16553.

22. A method of identifying a integrin modulating agent, the method comprising;
(a) providing a test cell population comprising a cell capable of expressing one or more nucleic acid sequences selected from the group consisting of FNX1-209 and 260;
(b) contacting the test cell population with a test agent;
(c) measuring expression of one or more of the nucleic acid sequences in the test cell population;
(d) comparing the expression of the nucleic acid sequences in the test cell population to the expression of the nucleic acid sequences in a reference cell population comprising at least one cell whose integrin modulating agent expression status is known; and
(e) identifying a difference in expression levels of the FNX sequence, if present, in the test cell population and reference cell population, thereby identifying an integrin modulating agent

23. The method of claim 22, wherein the method comprises comparing the expression of 200 or more of the nucleic acid sequences.

24. The method of claim 22, wherein the method comprises comparing the expression of 100 or more of the nucleic acid sequences.

25. The method of claim 22, wherein the method comprises comparing the expression of 25 or more of the nucleic acid sequences.

26. The method of claim 22, wherein the expression of the nucleic acid sequences in the test cell population is decreased as compared to the reference cell population.

27. The method of claim 22, wherein the expression of the nucleic acid sequences in the test cell population is increased as compared to the reference cell population.

28. The method of claim 22, wherein the test cell population is provided in vitro.

29. The method of claim 22, wherein the test cell population is provided ex vivo from a mammalian subject.

30. The method of claim 22, wherein the test cell population is provided in vivo in a mammalian subject.

31. The method of claim 22, wherein the test cell population is derived from a human or rodent subject.

32. The method of claim 22, wherein the test cell population includes a blood cell.

33. The method of claim 22, wherein the test cell population includes a monocyte.

34. A method of treating or preventing atherosclerosis in a subject, the method comprising administering to the subject an agent that modulates the expression or activity of one or more of the nucleic acids selected from the group consisting of FNX1-208 and 209.

35. A method of treating or preventing an inflammatory disorder in a subject, the method comprising administering to the subject an agent that modulates the expression or activity of one or more of the nucleic acids selected from the group consisting of FNX1-208 and 209.

36. A method of inhibiting monocyte differention, the method comprising contacting a cell with an agent that modulates the expression or activity of one or more of the nucleic acids selected from the group consisting of FNX1-208 and 209.

37. The method of claim 34, 35 or 36, wherein the agent is a is a FNX polypeptide, an nucleic acid encoding a FNX polypeptide, a FNX antibody, a FNX antisense nucleic acid or a nucleic acid that modulates the expression of a nucleic acid that encodes a FNX polypeptide.
